(19) United States
(12) Patent Application Publication
(10) Pub. No.: US 2016/0067175 A1
(43) Pub. Date: Mar. 10, 2016

(54) INTRATHECAL MULTIDRUG INFUSION FOR PAIN CONTROL

(71) Applicant: Gilbert BLAISE, Montreal (CA)

(72) Inventor: Gilbert BLAISE, Montreal (CA)

(21) Appl. No.: 14/845,546

(22) Filed: Sep. 4, 2015

Related U.S. Application Data
(60) Provisional application No. 62/045,750, filed on Sep. 4, 2014.

(51) Int. Cl. A61K 9/00; A61K 31/135; A61K 31/4168; A61K 31/485; A61K 31/445

(52) U.S. Cl. CPC A61K 9/0019; A61K 31/485; A61K 31/445; A61K 31/4168; A61K 31/135

(57) ABSTRACT

The present invention relates to an intrathecal (IT) formulation comprising an opioid, a Na channel blocker, an alpha2-receptor agonist, an opioid mu or delta receptor competitive antagonist and an intravenous anesthetic.

### CROSS-REFERENCE TO RELATED APPLICATIONS

This application claims priority of U.S. provisional application No. 62/045750, filed Sep. 4, 2014, the specification of which is hereby incorporated by reference.

### BACKGROUND

(a) Field

Achieving effective, durable, and safe pain relief, especially in aged patients and end-stage malignancies can be a clinical challenge (1-6). Despite multimodal systemic approaches with the least doses of drugs to decrease systemic side effects, the pain is often poorly controlled with unacceptable side effects in some patients.

### SUMMARY

We present an alternative method for managing elderly and cancer-related intractable pain while improving cognitive function, based on time-limited intrathecal (IT) infusion of a multi-drug analgesic mixture. Three old patients suffering from a poorly-controlled pain were treated. A mixture of bupivacaine 1 mg/ml, naloxone 0.02 ng/ml, ketamine 100 μg/ml, morphine 0.01 mg/ml and clonidine 0.75 μg/ml was infused via IT by an external pump. The pain was successfully controlled and cognitive functions greatly improved. Oral and parenteral opioids consumption reduced dramatically. Such an IT approach could be considered as an alternative modality for intractable pain relief in elderly or in malignancies.

In accordance with one embodiment of the present invention, there is provided an intrathecal (IT) formulation comprising:
an opioid;
a Na channel blocker;
an alpha2-receptor agonist;
an opioid mu or delta receptor competitive antagonist; and
an intravenous anesthetic.

The opioid may be selected from the group consisting of morphine, fentanyl, sufentanil, hydromorphone, tramadol, pentazocine, dianorphine, methadone, mépéridine, and butorphanol.

The Na channel blocker may be selected from the group consisting of bupivacaine, ropivacaine and lidocaine.

The alpha2-receptor agonist may be selected from the group consisting of clonidine, dexmedetomidine, fadolmidine, and tinazidine.

The opioid mu receptor competitive antagonist may be selected from the group consisting of naloxone, naltrexone and naltrindole.

The intravenous anesthetic may be selected from the group consisting of ketamine, propofol, tricyclic antidepressants such as amitryptilline, and other tricyclic medications, other antidepressants such as SSRI, and SNRI; gabapentine, pregabalin, Midazolam, Baclofen, and adenosine; other compounds acting on adenosine receptors (A1,A2,A3), neostigmine, magnesium, atropine, dexamethasone, ketorolac and other NSAID, octreotide, ziconitide and droperidol.

In accordance with another embodiment of the present invention, there is provided a method of pain control and/or cognitive functions improvements in a patient which comprises administering by intrathecal (IT) infusion the intrathecal (IT) formulation of the present invention at a rate of 0.5 to 5 ml/h.

In accordance with another embodiment of the present invention, there is provided a kit to control pain and/or improve cognitive functions in a patient which comprises:

an intrathecal (IT) catheter optionally connected to a PORT-A-CATH ™, and

a reservoir for receiving a formulation and wherein the reservoir is to be connected to the intrathecal (IT) catheter.

### BRIEF DESCRIPTION OF THE DRAWINGS

Further features and advantages of the present disclosure will become apparent from the following detailed description, taken in combination with the appended drawings, in which:

FIG. 1 illustrates the stability of the drugs in the prepared solution at room temperature during five days measured by tandem mass spectrometry. Solution contains bupivacaine 1mg/ml, ketamine 100 μg/ml, morphine 0.01 mg/ml in NaCl 0.9% 50 ml plastic bag. The concentration of ketamine and bupivacaine is stable but morphine's concentration decreases 15% on first day and then stays mostly unchanged during the following days.

### DETAILED DESCRIPTION

To explore the efficacy of an alternative method to manage pain based on time-limited intrathecal (IT) infusion of an analgesic medication mixture, three patients with the ages of 69, 64 and 94 y suffering from intractable and poorly-controlled pain due to bed sores, pelvic metastatic mass and thoracic vertebra and ribs fractures, respectively, were treated. Daily dose of opioids could not be increased due to side effects. An intrathecal catheter 20G was placed by percutaneous approach in lumbar area while advancing toward thoracic region tunneled and fixed subcutaneously. It was connected to an external infusion pump with a mixture of bupivacaine 1 mg/ml, naloxone 0.02 ng/ml, ketamine 100 μg/ml, morphine 0.01 mg/ml and clonidine 0.75 μg/ml. Starting rate was 1 ml/h. The pain was mostly controlled with a rate of less than 1 ml/h. Opioid consumption was reduced dramatically. Catheter was kept for one month in first and third patient and for 6 months in the second one until his death. We did not observe major side effects such as hypotension, constipation, muscle weakness, sphincter dysfunction and cognitive or mood deterioration with this approach. One patient had urinary tract infection followed by sepsis and meningitis which was easily cured by antibiotics. Catheter was removed in this patient. Intrathecal infusion with a low-concentration multi-drugs mixture could be considered as an alternative modality for intractable pain relief in elderly or malignancies. The cognitive functions of all patients were also greatly improved.

Three patients admitted in hospital became candidates of IT infusion (Table-1).

TABLE 1
Comparison of opioid consumption before
and after intrathecal catheter insertion

| | | | | | | Maximal |
|---|---|---|---|---|---|---|
| | | | | Equivalent | Equivalent | inpatient |
| | | | | oral | oral | IT |
| | | | | morphine | morphine | infusion |
| | Age | | Cause of | before IT | after IT | rate |
| Patient | (years) | sex | pain | (mg/d) * | (mg/d) * | (ml/h) |
| 1 | 69 | F | Bed sore | 220 | 30 | 1 |
| 2 | 64 | M | Pelvic | 375 + | 60 | 2.5** |
| | | | metastatic | methadone | | |
| | | | cancer | 24 mg/d | | |
| 3 | 94 | F | Ribs and | 200 | 30 | 1 |
| | | | thoracic | | | |
| | | | vertebra | | | |
| | | | fracture | | | |

* Calculated on the basis of opioid equianalgesic dose conversion table (Montreal University Hospital-CHUM)
**In this patient, the concentration of drugs were modified to avoid passing maximum intrathecal daily dose of bupivacaine

IT Intrathecal; F female; M male

Case 1: A 69 years old female, with history of laryngeal neoplasia, diabetes and stroke (with hemiparesis) was admitted in hospital for dysphagia and large painful sacral bed sore. Pain was intractable and difficult to control by conventional pain treatment. At the time of admission, she was using gabapentin 2400 mg/day, acetaminophen 3-4 g/day and 25 μg/h fentanyl patch every three days. We increased fentanyl patch to 50 μg/h and prescribed parenteral morphine as breakthrough dose. A total oral-equivalent morphine daily dose of 220 mg was unsuccessful to control the pain. Due to side effects, it was not possible to increase opioid dose.

Case 2: A 64 years old man with history of end-stage adenocarcinoma of rectum with metastasis to liver and pelvis, sacral plexus involvement due to huge pelvic metastatic mass as 10*14*7 cm was suffering from an excruciating pelvic and sacral pain which was refractory to usual pharmacologic approach (oral or parenteral opioids). Treatment of 30 mg/d parenteral hydromorphone plus 24 mg/d methadone (while resulting in adverse reactions such as hallucination and drug interaction) was unable to control patient's pain.

Case 3: A 94 years old female with multiple thoracic vertebra and ribs fracture (confirmed fracture on T9 without neurological compromise, left 3^{rd} to 6^{th} ribs fracture and suspected fracture on T7, T8, T10 and T11 from imaging analysis) due to falling down, with intractable pain which had made patient bedridden. The pain was difficult to control by conventional pain treatment. Attempt was made to control the pain by prescribing hydromorphone up to 10-15 mg orally or parenterally per day, acetaminophen 4 g/day, celecoxib 200 mg/day, and fentanyl patch up to 50 μg/h every three days. Despite high dose opioid and remarkable side effects, pain was not successfully controlled. Severe pain made the patient unable to exit from complete bedridden situation.

Due to inability to control the pain and remarkable side effects and after discussing with the patients and the attending physicians, we decided to try IT approach for pain management. We obtained an informed consent from the patients. In all three patients, an IT catheter 20G was placed by percutaneous approach in lumbar area (L2-L3 or L3-L4) advancing to thoracic region and tunneled and fixed subcutaneously. It was connected to an external infusion pump with a mixture of bupivacaine 1mg/ml, naloxone 0.02 ng/ml, ketamine 100 μg/ml, morphine 0.01 mg/ml and clonidine 0.75 μg/ml. All of them were preservative—free except naloxone which was not available in the market at that time. Starting rate was 1 ml/h. In all of the patients, pain was successfully controlled and oral or parenteral opioid requirements decreased significantly. Acetaminophen was continued as 3-4 g/day in all of them. Infusion rate was adjusted during the following days according to patients' requirement and pain relief (on the basis of visual analog scale in rest time and normal daily activities).

Results: In the first patient, following the catheter insertion and an infusion rate of 1 ml/h, pain decreased dramatically as parenteral morphine was stopped and fentanyl patch was decreased to 12 μg/h. Parenteral hydromorphone was considered as breakthrough during bed sore dressing. Patient analgesic requirement stayed stable with an infusion rate of 1 ml/h or less. The infusion bag was changed every 24 hours. Meanwhile, bed sore healed almost completely. After one month of this regimen, following a fever episode, a sepsis work up was done that showed a urinary and cerebrospinal fluid (CSF) reaction. In spite of immediate parenteral antibiotics, the fever continued. So the catheter was removed. Klebsiella pneumoniae was detected in urine and CSF culture. Surprisingly, despite catheter removal, the patient did not request pain medication more than before. She left hospital in good condition without any sequels.

In the second patient, pain was not successfully controlled by parenteral hydromorphone 30 mg/day, methadone 24 mg/day and acetaminophen 3 g/day. Due to opioids and co-analgesics side effects (such as delirium), it was not possible to either increase dosage or to add any other agent. Patient had a history of allergy to morphine. So, we prepared the mixture with equivalent dose of preservative-free hydromorphone (a hydromorphine-morphine ratio of 1 to 5). Following catheter insertion, pain dramatically decreased with an infusion rate of 1 ml/h. Hydromorphone was used as breakthrough pain control. Due to patient's need, infusion rate was increased to 2.5 ml/h, but to avoid passing the limit of intrathecal bupivacaine daily dose, we changed the mixture as follows: hydromorphone 3 μg/ml, clonidine 1.25 μg/ml, naloxone 2 ng/ml, ketamine 100 μg/ml and bupivacaine 0.7 mg/ml. The infusion bag was changed every 24 hours. At this point, patient occasionally requested hydromorphone. Methadone was decreased and stopped during the following days. Infusion rate was gradually decreased to 1 ml/h and patient left hospital in good condition. For sterility concern, we increased the drugs' concentration during outpatient period. That let us to decrease infusion rate (having the same daily dose) and to change the bag every 72 hours. The catheter was kept in place up to five months out of hospital under the supervision of a family physician. The patient died of his cancer after five months. During the last days of his life, we had to increase the infusion rate to control the pain and make a comfortable state in terminal phase.

In the third patient, the pain decreased dramatically as we no longer had to use hydromorphone (it was prescribed whenever needed but patient never requested). As supplement, 12 μg/h fentanyl patch every three days and topical lidocaine were used. Infusion rate was gradually decreased to 0.3 ml/hour due to patient pain relief and satisfaction (on the basis of visual analog scale in rest time and normal daily activities) and finally stopped after 2 months. The infusion was stopped at this point and catheter was removed. There was a local irritation in catheter exiting point without any systemic manifestation which was successfully controlled by local care. Patient left hospital in good condition.

There were no remarkable side effects such as catheter's insertion site infection, constipation, muscle weakness, sphincter dysfunction and cognitive or mood deterioration in our patients. Chemical stability testing of the drug mixture was assessed by the Biochemistry Department during a five day period at room temperature. The appearance of the solution was monitored by visual inspection and the drug concentrations were quantified by liquid chromatography coupled to tandem mass spectrometry (LC-MS/MS). No change in appearance was observed during the five day period. The ketamine and bupivacaine concentrations did not change over time. Morphine concentration decreased by approximately 15% on the first day and remained relatively steady during the following days (FIG.-1).

Pain management is a great challenge in elderly and end-stage malignancies. Age-related co-morbidities, polymedication and physiologic changes in elderly limit liberal use of systemic opioids and NSAIDS prescription (7). In neuroaxial analgesia, IT has less undesirable drug-related side effects due to fewer doses. Moreover, the evidence shows better results for IT approach in comparison with epidural approach in cancer-related pain (2B+and 2C+respectively) (8). Morphine, baclofen and ziconotide are the only FDA-approved medications for IT use. Apart from bupivacaine, the others have been used successfully via IT approach at a greater dose than the one we used in our patients (Table-2).

TABLE 2
Comparison of recommended intrathecal
daily drugs' doses and our doses

| | | |
|---|---|---|
| Product | Recommended dose (9) | Our dose* |

| | | | | |
|---|---|---|---|---|
| Morphine | 1-20 | mg | 0.08-0.24 | mg |
| Bupivacaine | 4-30 | mg | 8-24 | mg |
| Clonidine | 30-1000 | μg | 6-18 | μg |

| | | | |
|---|---|---|---|
| Naloxone | no data available | 0.2-0.5 | ng |

| | | | | |
|---|---|---|---|---|
| Ketamine | 1-50 | mg | 1-2.4 | mg |

*Calculated on the basis of ≦1 ml/h infusion rate

In our drugs mixture, morphine, hydromorphone, bupivacaine and clonidine are the drugs that have been already used successfully by IT method (mode) (9). In chronic pain syndromes, morphine is the opioid of first choice for IT administration. It has hydrophilic properties, so, morphine will spread more than fentanyl (and sufentanil) after IT administration and thus extend the area of analgesia. Its presynaptic and postsynaptic effects are via G-protein-linked opioid mu (mainly), delta and kappa receptors. Presynaptic interaction inhibits the release of substance-P and calcitonin gene-related peptide by means of interactions with N-type voltage-dependent calcium channels and reduced calcium influx. Postsynaptic activation of opioid receptors leads to inhibition of adenylate cyclase and also results in opening of potassium channels which, in turn causes hyperpolarization, rendering the postsynaptic second-order neuron less responsive (10).

Local anesthetics effect is via blocking the Na channels of neuronal tissue and disrupts pain transmission. In the clinical setting, intrathecal bupivacaine dose ranges from 3 to 50 mg/d (9). The recommended dose of bupivacaine combined with opioids is between 1-14 mg/day and up to more than 30 mg/day did not cause bladder dysfunction or muscle weakness (10). On the other hand, a dose up to more than 100 mg/day has been reported (11). In our cases, two patients usually received ml/h infusion rate which was equivalent to ≦24 mg/day. The maximum inpatient dose of bupivacaine for the second patient was 42 mg/day.

Clonidine is an alpha2-receptor agonist which is located in both presynaptic and postsynaptic neurons. It reduces presynaptic calcium entry and an increased potassium influx post-synaptically, which initiates hyperpolarization of the postsynaptic cell membrane. Combined with local anesthetics and morphine, it is reported to have a synergistic action for pain relief (9). IT administration of clonidine (average daily dose range from 50 to 200 μg) reduces the risk of morphine tolerance and thus lessens the risk for opioid-related adverse effects due to dose escalation (10). Side effects include dry mouth, sedation, bradycardia and hypotension. Sudden discontinuation of long-term IT therapy may lead to rebound hypertension, panic attacks, and psychotic behavior (12).

Naloxone is an opioid mu-receptor competitive antagonist, but in low dose (in fact “ultra-low dose”), it helps controlling pain and prevents from hyperalgesia (13). Review of literature suggests that under certain conditions, low-dose opioid antagonists (alone or in combination with opioids) can produce an antinociceptive or analgesic response (14). Furthermore, they have been used successfully in Crohn's disease and IBS (irritable bowel syndrome) to control disease-associated pain. The possible mechanisms might be upregulation of opioid receptors, increased levels of endogenous opioids, decreased opioid receptor coupling to stimulatory G-proteins (mediated through filamin A) and inhibition of opioid agonist-induced activation of glial cells (14). In a recent study by Mattioli et al., it was demonstrated that co-administration of morphine with ultra-low-dose naltrexone attenuates gliosis in rats, which was noted by an attenuation of increase of the glial proteins GFAP and CD3/CD11B, increase of astrocyte cell volume and astrocyte proliferation (15). Their IT use alone or in combination with opioids could have analgesic effect, as shown in animal models (16). In an animal model study, ultra-low dose naloxone enhanced the antihyperalgesia and antiallodynia effects of morphine in rats, possibly by reducing TNF-α and TNFR1 expression and excitatory amino acids (EAAs) such as glutamate and aspartate concentrations in spinal dorsal horn (17). In a case report, adding 50 ng/day naloxone to IT morphine infusion dramatically enhanced morphine analgesic effect, without apparent side effects for more than 3 years (18). These mechanisms can explain partly the analgesic effect of our mixture. The mechanistic rationale for naloxone/morphine dose and concentration ratio was on the basis of animal studies and a case report (18) in which an IT naloxone/morphine concentration ratio of 1/10⁵ was efficient to control the pain for three years. One potential concern is remote possibility of naloxone neurotoxicity. It is not approved for IT use but its extremely low concentration, molecular characteristics and animal studies could make it safe for IT use. Molecular structure of naloxone is virtually identical to morphine except in minor moiety substitution at position 9 (or 13). Further, IT naloxone plus morphine in rodents, have not demonstrated signs of spinal cord toxicity (16,19). Even, some studies show neural protection of naloxone against ischemic situations (20). Due to chemical characteristics, ultra-low concentration and animal studies, it seems unlikely that IT naloxone would produce spinal cord toxicity. At the time of our (current) study, naloxone was not available as a formulation without preservative.

Ketamine is an intravenous anesthetic which has analgesic effect in sub-anesthetic doses. Besides, it has anti-hyperalgesic effect due to the impact on NDMA receptors (21) and anti-allodynic effect by suppressing TLR (toll-like receptor)-mediated signal transduction. (22). It could be used as IT approach in end-stage cancer related pain (23). Ketamine possesses a plethora of other actions that enhance its analgesic properties. These include blocking non-NMDA glutamate and muscarinic cholinergic receptors, facilitating GABA-A signaling, weakly binding to opioid receptors, and possessing local anesthetics and possibly neuroregenerative properties (9). It has been used successfully to control cancer and non-cancer pain. But the main concern is possible neurotoxic effect during long-term IT use (even with preservative-free S (+) enantiomer). In one recent animal study, IT injection of a large dose of 1mg/kg in dogs had no histological alterations of spinal tissue or meninges (24). Besides, there is more evidence which shows that IT infusion of ketamine would be useful in end-stage cancer related pain (23). Despite some studies reporting neurotoxicity following IT ketamine (25,26), our used dose is much less than the one in these reports and as mentioned previously, the other studies do not show neurotoxic effects in animal models (24). On the other hand, we should not neglect its beneficial effects in inflammatory process and post-operative outcome. In a recent article, De Kock et al (27) reviewed ketamine effect on inflammatory process. They concluded that ketamine is an immunomodulator which prevents the exacerbation and the extension of local inflammation without blunting the local process and delaying inflammatory resolution. This can explain some beneficial aspects of ketamine in post-operative outcome and cognitive dysfunction. Moreover, repeated high dose ketamine might have neurotoxic effect in immature brains in the absence of noxious stimuli whereas it may be neuroprotective in the same brains in the presence of strong painful stimuli (28). Controversy continues about IT ketamine, but utilizing low-dose preservative-free ketamine in elderly and end-stage malignancies suffering from intractable pain would be a reasonable choice in pain management.

Comparison of systemic opioids consumption (oral and parenteral) before and after catheter insertion shows a significant reduction in daily dose of opioids in our patients (Table-2). The equivalent dose of IT morphine could not explain this reduction. Although there is no consensus about IT to oral opioid conversion rate (29), compared to maximum conversion ratio calculated by Krames (30), our doses after catheter insertion are much less than the dose used before. Direct IT infusion, synergistic effect and different mechanisms of action could explain sufficient analgesic effect of our drug combination even at lower concentrations. Due to very low concentration of the drugs (especially ultra-low dose for naloxone), absorption and systemic effect could not be very important in analgesic effect of the mixture.

To achieve a comfortable pain relief in the second patient, we needed larger doses during the short period of admission in hospital and during last days of his life. Severe metabolic changes due to malignancy, progressive tumor invasion, possible tolerance and local aggravated inflammatory processes which interact with prescribed medications' effects could explain a part of this phenomenon.

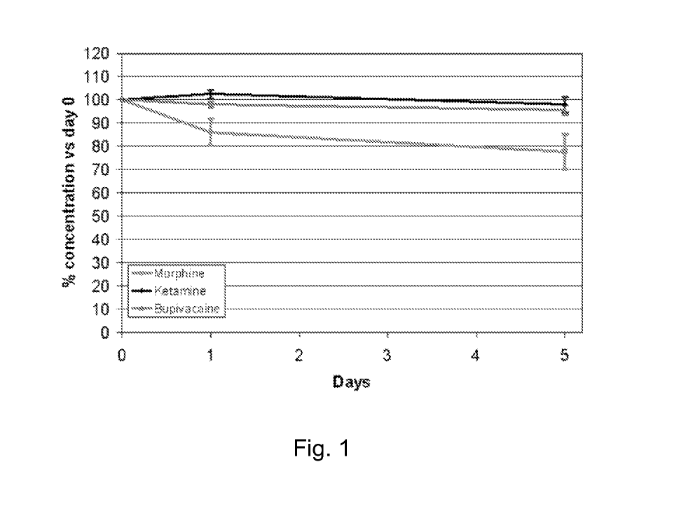

Physicochemical stability of a drug admixture is assumed if the physical appearance of a solution does not change and drug concentration stays stable. Stability is assumed for an ingredient if the loss is less than 10% of the initial concentration after a period of 96 hours. Our stability assessment shows that bupivacaine and ketamine are sufficiently stable during five days at room temperature (FIG. 1). There is 15%-20% reduction in morphine concentration which may be related to its very low concentration or the other factors. This value is under the critical value for stability but did not decrease further. The infusion bag was changed every 24 hours during hospitalization (inpatient) and every 72 hours at home. No loss of clinical efficacy was observed. It was not possible to effectively assess the stability of naloxone or clonidine at these low concentrations using our LC-MS assay. Meanwhile, their effect on pain reduction is explained by their ultra-low dose concentration. A more precise, accurate and sensitive method for separation and quantification of active ingredients is under development.

Unfortunately, even in IT method for pain management, there are always drug-related complications such as constipation, muscle weakness, sphincter dysfunction and cognitive or mood deterioration. However, we did not observe these side effects as major complications, probably due to positive interaction between the drugs and very low daily dose and concentration.

Achievement of satisfying pain control without major complications by a rate of less than 1 ml/h in our patients could suggest this method as an alternative in elderly patients whose pain could not be well-controlled by conventional opioids. To determine the kind and the dose of probable drugs in mixture, we need more detailed studies with more patients. The main beneficial effect of a drug admixture infusion is to avoid multiple injections. It decreases the risk related to line manipulation, confusion with route of administration, simplify the drug regimen and in some circumstances, facilitates to discharge patient from hospital to care at home. The other advantage of our tested mixture in low concentration and lower daily doses is lower risk of neurotoxicity. But this advantage is not so great compared to the importance of pain control in elderly and end-stage cancer related pain. Having greater number of patients and conducting randomized trials could lead to find more appropriate drug combination.

Pain can be better controlled by multi-drug inthrathecal infusion for a short time in advanced-age and end-stage malignancies. The advantages of this method are the better pain control and less systemic side effects due to lower prescribed doses.

While preferred embodiments have been described above and illustrated in the accompanying drawings, it will be evident to those skilled in the art that modifications may be made without departing from this disclosure. Such modifications are considered as possible variants comprised in the scope of the disclosure.

### REFERENCES:

1—Institute of Medicine. Relieving pain in America: A blueprint for transforming prevention, care, education, and research.
http://www.iom.edu/˜/media/Files/Report%20Files/2011/Relieving-Pain-in-America-A-Blueprint-for-Transforming-Prevention-Care-Education-Research/Pain%20Research%202011%20Report%20Brief.pdf.
Accessed Jun. 13, 2013.
2—Deandrea S, Montanari M, Moja L, Apolone G. Prevalence of undertreatment in cancer pain. A review of published literature. Ann Oncol 2008; 19:1985-91.
3—Costantini M, Ripamonti C, Beccaro, et al. Prevalence, distress, management, and relief of pain during the last 3 months of cancer patients' life. Results of an Italian mortality follow-back survey. Ann Oncol 2009; 20:729-35.
4—Breivik H, Cherny N, Collett B, et al. Cancer-related pain: a pan-European survey of prevalence, treatment, and patient attitudes. Ann Oncol 2009; 20:1420-33.
5—Apolone G, Corli O, Caraceni A, et al. Cancer Pain Outcome Research Study Group (CPOR SG) Investigators. Pattern and quality of care of cancer pain management. Results from the Cancer Pain Outcome Research Study Group. Br J Cancer 2009; 100:1566-74.
6—Hong S H, Roh S Y, Kim S Y, et al. Change in cancer pain management in Korea between 2001 and 2006: results of two nationwide surveys. J Pain Symptom Manag 2011; 41:93-103
7—Atkinson T J, Fudin J, Pandula A, Mirza M. Medication Pain Management in the Elderly: Unique and Underutilized Analgesic Treatment Options. Clin Ther. 2013; 35:1669-89.
8—Vissers K C, Besse K, Wagemans M, et al. Pain in patients with cancer. Pain Pract 2011; 11:453-75.
9—Cohen S P, Dragovich A. Intrathecal analgesia. Med Clin North Am 2007; 91:251-70.
10—Ver Donck A, Vranken J H, Puylaert M, Hayek S, Mekhail N, Van Zundert J. Intrathecal Drug Administration in Chronic Pain Syndromes. Pain Pract 2013 Oct. 4. doi: 10.1111/papr.12111. [Epub ahead of print]
11—Van Dongen R T, Crul B J, De Bock M. Long-term intrathecal infusion of morphine and morphine/bupivacaine mixtures in the treatment of cancer pain: a retrospective analysis of 51 cases. Pain 1993; 55:119-23.
12—Ghafoor V L, Epshteyn M, Carlson G H, Terhaar D M, Charry O, Phelps P K. Intrathecal drug therapy for long-term pain management. Am J Health Syst Pharm 2007; 64:2447-61.
13—Aguado D, Abreu M, Benito J, Garcia-Fernandez J, Gomez de Segura I A. Effects of naloxone on opioid-induced hyperalgesia and tolerance to remifentanil under sevoflurane anesthesia in rats. Anesthesiology 2013; 118:1160-9.
14—Taylor R Jr, Pergolizzi J V Jr, Porreca F, Raffa R B. Opioid antagonists for pain. Expert Opin Investig Drugs 2013; 22:517-25.
15—Mattioli T A, Milne B, Cahill C M. Ultra-low dose naltrexone attenuates chronic morphine-induced gliosis in rats. Mol Pain 2010; 6:22.
16—Lunzer M M, Yekkirala A, Hebbel R P, et al. Naloxone acts as a potent analgesic in transgenic mouse models of sickle cell anemia. Proc Natl Acad Sci USA 2007; 104:6061-5.
17—Yang C P, Cherng C H, Wu C T, et al. Ultra-Low Dose Naloxone Enhances the Antihyperalgesic Effects of Morphine and Attenuates Tumor Necrosis Factor-α and Tumor Necrosis Factor-α Receptor 1 Expression in the Dorsal Horn of Rats with Partial Sciatic Nerve Transection. Anesth Analg 2013; 117:1493-502.
18—Hamann S, Sloan P A, Witt W. Low-dose intrathecal naloxone to enhance intrathecal morphine analgesia: a case report. J Opioid Manag 2008; 4:251-4.
19—Ru-Yin Tsai, Fong-Lin Jang, Yueh-Hua Tai1, Shinn-Long Lin, Ching-Hui Shen and Chih-Shung Wong. Ultra-Low-Dose Naloxone Restores the Antinociceptive Effect of Morphine and Suppresses Spinal Neuroinflammation in PTX-Treated Rats. Neuropsychopharmacology 2008; 33:2772-82.
20—Hsu Y Y, Jong Y J, Lin Y T, Tseng Y T, Hsu S H, Lo Y C. Nanomolar Naloxone Attenuates Neurotoxicity Induced by Oxidative Stress and Survival Motor Neuron Protein Deficiency. Neurotox Res 2014; 25:262-70.
21—Fisher K, Coderre T J, Hagen N A. Targeting the N-methyl-D-aspartate receptor for chronic pain management. Preclinical animal studies, recent clinical experience and future research directions. J Pain Symptom Manage 2000; 20:358-73.
22—Mei X P, Zhou Y, Wang W, et al. Ketamine depresses toll-like receptor 3 signaling in spinal microglia in a rat model of neuropathic pain. Neurosignals 2011; 19:44-53.
23—Mion G, Marchetti F, Samama C M. Compassionate use of intrathecal ketamine for intractable cancer pain (Lett). Ann Fr Anesth Reanim 2013; 32:621-2.
24—Rojas A C, Alves J G, Moreira E Lima R, et al. The effects of subarachnoid administration of preservative-free S(+)-ketamine on spinal cord and meninges in dogs. Anesth Analg 2012; 114:450-5.
25—Karpinski N, Dunn J, Hansen L, et al. Subpial vacuolar myelopathy after intrathecal ketamine: report of a case. Pain 1997;73:103-5.
26—Stotz M, Oehen H P, Gerber H. Histological findings after long-term infusion of intrathecal ketamine for chronic pain: a case report. J Pain Symptom Manage 1999; 18:223-8.
27—De Kock M, Loix S, Lavand'homme P. Ketamine and peripheral inflammation. CNS. Neurosci Ther 2013; 19:403-10.
28—Yan J, Jiang H. Dual Effects of Ketamine: Neurotoxicity Versus Neuroprotection in Anesthesia for the Developing Brain. J Neurosurg Anesthesiol 2014; 26:155-60.
29—Sylvester R K, Lindsay S M, Schauer C. The conversion challenge: from intrathecal to oral morphine. Am J Hosp Palliat Care. 2004; 21:143-7.
30—Krames E S: Intraspinal opioid therapy for chronic nonmalignant pain: Current practice and clinical guidelines. J Pain Symptom Manage 1996; 11:333-52.

1. An intrathecal (IT) formulation comprising:
an opioid;
a Na channel blocker;
an alpha2-receptor agonist;
an opioid mu or delta receptor competitive antagonist; and
an intravenous anesthetic.

2. The intrathecal (IT) formulation of claim 1, wherein the opioid is selected from the group consisting of morphine, fentanyl, sufentanil, hydromorphone, tramadol, pentazocine, dianorphine, methadone, mépéridine, and butorphanol.

3. The intrathecal (IT) formulation of claim 1, wherein the Na channel blocker is selected from the group consisting of bupivacaine, ropivacaine and lidocaine.

4. The intrathecal (IT) formulation of claim 1, wherein the alpha2-receptor agonist is selected from the group consisting of clonidine, dexmedetomidine, fadolmidine and tinazidine.

5. The intrathecal (IT) formulation of claim 1, wherein the opioid mu receptor competitive antagonist is selected from the group consisting of naloxone, naltrexone and naltrindole.

6. The intrathecal (IT) formulation of claim 1, wherein the intravenous anesthetic is selected from the group consisting of ketamine, propofol, tricyclic antidepressants such as amitryptilline, and other tricyclic medications, other antidepressants such as SSRI, and SNRI; gabapentine, pregabalin, Midazolam, Baclofen, and adenosine; other compounds acting on adenosine receptors (A1,A2,A3), neostigmine, magnesium, atropine, dexamethasone, ketorolac and other NSAID, octreotide, ziconitide and droperidol.

7. A method of pain control and/or cognitive functions improvements in a patient which comprises administering by intrathecal (IT) infusion the intrathecal (IT) formulation of claim 1 at a rate of 0.5 to 5 ml/h.

8. A kit to control pain and/or improve and/or cognitive functions in a patient which comprises:
an intrathecal (IT) catheter optionally connected to a PORT-A-CATH™; and
a reservoir for receiving a formulation and wherein the reservoir is to be connected to the intrathecal (IT) catheter.
